(19) United States
(12) Patent Application Publication
(10) Pub. No.: US 2014/0228757 A1
(43) Pub. Date: Aug. 14, 2014

(54) BENT SYRINGES AND DEVICES INCLUDING SAME

(71) Applicant: MEDRAD, INC., Indianola, PA (US)

(72) Inventor: Mark TROCKI, Cheswick, PA (US)

(73) Assignee: MEDRAD, INC., INDIANOLA, PA (US)

(21) Appl. No.: 13/764,505

(22) Filed: Feb. 11, 2013

(51) Int. Cl. A61M 5/31

(52) U.S. Cl. CPC A61M 5/3135
USPC 604/131; 604/218; 604/236

(57) ABSTRACT

Bent syringes, mounting systems for bent syringes, and devices incorporating bent syringes are described herein. The syringes include a bent cylindrical body bent about a radius and a plunger bent about the radius of the bent cylindrical body and configured and arranged to be received by the proximal open end of the bent cylindrical body and movably disposed within at least a portion of the bent cylindrical body.

### BACKGROUND

Syringes typically include a cylindrical body configured to house a liquid and a plunger configured to be inserted into the cylindrical body and provide expulsion force as the plunger progresses through the cylindrical body causing the contents of the body to be expelled through an orifice at the distal end of the body. Such linear syringes have found use as handheld manual injectors and in numerous devices where actuators provide expulsion force to plunger. Injector-actuated syringes and powered injectors are used in medical procedures such as angiography, computed tomography, ultrasound and NMR/MRI. Typically, such injectors include drive members including a piston connected to a syringe plunger. These drive members provide a linear compression to the plunger. Therefore, the drive member and piston must have a range of movement at least equal to the length of the cylindrical body. As such, for high volume syringes, the syringe, plunger, and drive mechanisms must be long and bulky.

### SUMMARY OF THE INVENTION

Various embodiments are directed to syringes including a bent cylindrical body and a plunger bent about the radius of the bent cylindrical body configured and arranged to be received by the proximal open end of the bent cylindrical body and movably disposed within at least a portion of the bent cylindrical body. The cylindrical body of the bent syringes are, generally about a radius and have a proximal open end and a distal conical end. The conical end generally includes at least one discharge outlet.

Other embodiments are directed to mounting systems for bent syringes and devices that incorporate the bent syringes. The devices of such embodiments may include a removable syringe having a bent cylindrical body with a proximal open end and a distal conical end with at least one discharge outlet and a plunger having curvature substantially similar to the bent cylindrical body and being configured and arranged to be received by the proximal open end of the bent cylindrical body and movably disposed within at least a portion of the cylindrical body; and a hinge operably connected to the bent cylindrical body and the plunger, the hinge being disposed at an intersection of a radius of the bent cylindrical body and a radius of the plunger. A spindle may be configured and designed to be received by the hinge and operably connected to one of the bent cylindrical body or the plunger, and a drive motor may be operably connected to the spindle and being capable of providing rotational force to the spindle.

### BRIEF DESCRIPTION OF THE DRAWINGS

In the following detailed description, reference is made to the accompanying drawings, which form a part hereof. In the drawings, similar symbols typically identify similar components unless context dictates otherwise. The illustrative embodiments described in the detailed description, drawings, and claims are not meant to be limiting. Other embodiments may be utilized and other changes may be made, without departing from the spirit or scope of the subject matter presented herein. It will be readily understood that the aspects of the present disclosure, as generally described herein and illustrated in the Figures, can be arranged, substituted, combined, separated, and designed in a wide variety of different configurations, all of which are explicitly contemplated herein.

FIG. 1A is a drawing of an exemplary bent syringe showing portions of the cylindrical body, plunger, and hinge.

FIG. 1B is a drawing of an exemplary bent cylindrical body showing overlaid geometric elements.

FIG. 1C is a drawing of an exemplary bent syringe including a spacer and small diameter cylindrical body.

FIG. 1D is a drawing of an exemplary bent syringe having a molded hinge in open configuration.

FIG. 1E is a drawing of an exemplary bent syringe having a molded hinge in folded configuration.

FIG. 2A is a drawing showing a bent syringe attached to a handle for manual dispersion of materials from the bent syringe.

FIG. 2B is a drawing showing exemplary elements of the handle for manual dispersion of materials from a bent syringe.

FIG. 3A is a drawing showing a bent syringe removable attached to an exemplary mounting system.

FIG. 3B is a drawing showing a mounting system without an attached bent syringe.

FIG. 3C is a drawing showing a the profile of a mounting system associated with a spindle of a drive motor for rotating the plunger of a bent syringe.

FIG. 4A is a drawing of a mounting system including a groove through which a spindle of a drive motor attaches to a bent syringe.

FIG. 4B is a drawing of the rear of a mounting system including a groove through which a spindle of a drive motor attaches to a bent syringe.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this document is to be construed as an admission that the embodiments described in this document are not entitled to antedate such disclosure by virtue of prior invention.

This disclosure is not limited to the particular systems, devices and methods described, as these may vary. The terminology used in the description is for the purpose of describing the particular versions or embodiments only, and is not intended to limit the scope.

The word “proximal” refers to a direction relatively closer to a clinician using the device described herein, and the word “distal” refers to a direction relatively further from the clinician. For example, the end of a catheter placed within the body of a patient is considered a distal end of the catheter, while the catheter end remaining outside the body is a proximal end of the catheter.

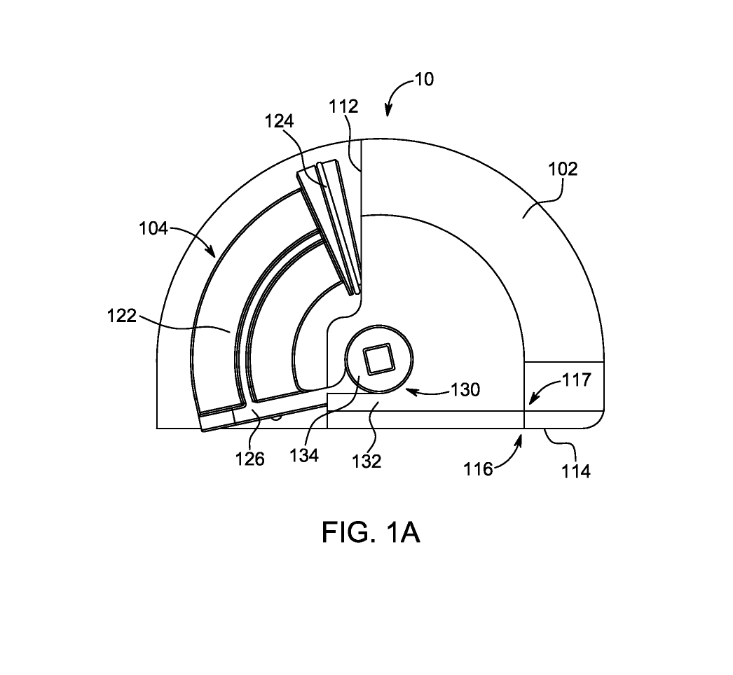

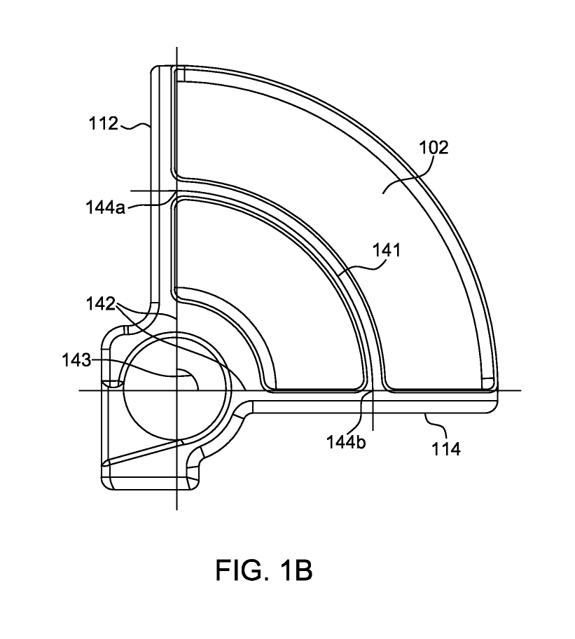

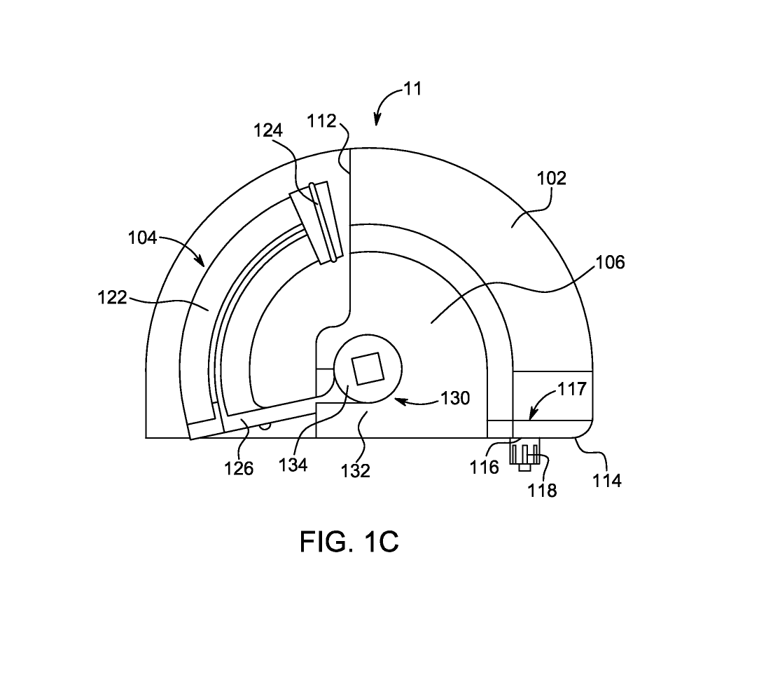

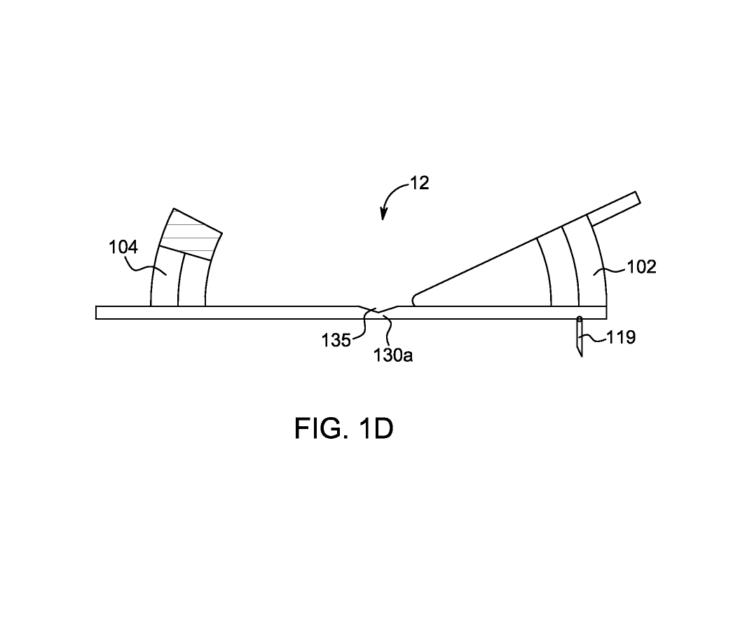

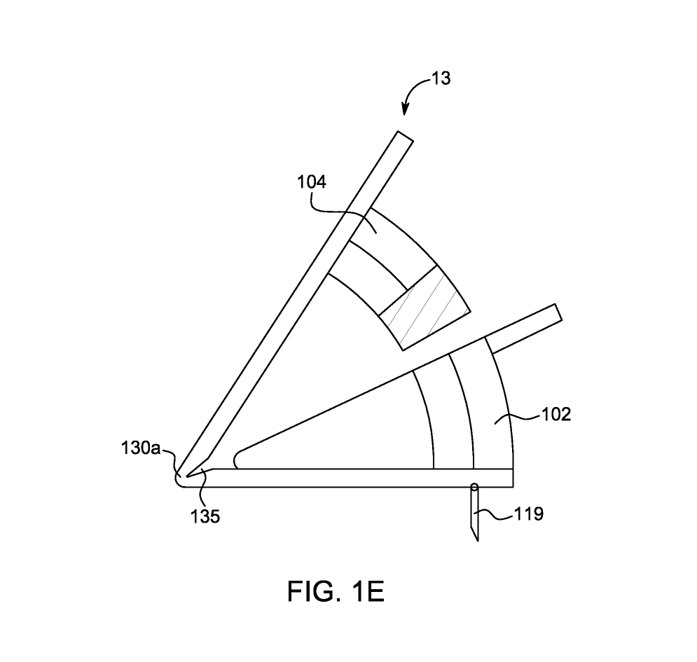

Embodiments of the invention include syringes having a generally cylindrical body that is bent about a radius to produce a bent syringe body and a bent plunger designed to fit within the bent syringe body and devices including such bent syringes. An exemplary bent syringe 10 is provided in FIG. 1A and shows a generally bent cylindrical body 102 having about a 90° bend radius. FIG. 1A further shows a plunger 104 including a piston 122 and a stopper 124 bent at about a 90° bend radius and configured and arranged to be received by the proximal open end 112 of the bent cylindrical body 102 to be movably disposed within at least a portion of the bent cylindrical body 102. The bent cylindrical body 102 may include a bent axial bore extending from the open proximal end 112 to a substantially closed distal end 114 including at least one outlet port 116. The plunger 104 may have a curvature matching the curvature of the bent cylindrical body 102 and the stopper 124 may be sized to fit within and contact an inner diameter of the axial bore of the bent cylindrical body 102.

FIG. 1B, shows a bent cylindrical body 102. An arc 141 can be formed at the center axis of the bent cylindrical body having a proximal end point 144a and a distal end point 144b, which correspond to the proximal end 112 and the distal end 114, respectively, of the bent cylindrical body 102. The bend angle 143 is defined as the angle created at the intersection of radii of curvature 142 that extend from the proximal and distal end points 144a, 144b to the point of their intersection. In various embodiments, the cylindrical body may be bent about a radius such that the radii of curvature remains constant over the entire length of the cylindrical body thereby providing a cylindrical body that has a smooth arced shape.

The bent cylindrical body 102 may have a bend angle of up to about 180°. For example, the angle of rotation may be from about 30° to about 180°, from about 45° to about 100° or any angle of rotation between these exemplary ranges. In particular embodiments, the angel of rotation may be about 90° as illustrated in FIG. 1A. The bent cylindrical body 102 may have any size and bent syringe bodies having volumes similar to those sold commercially can be created by varying the diameter of the bent cylindrical body 102, the length of a radius from the hinge 130 to the bent cylindrical body 102, or combinations thereof. For example, syringes 11 of equal volume can be created by providing a bent cylindrical body 102 having a large diameter bore or providing bent cylindrical body 102 having a smaller diameter that is space from the hinge 130. Thus, in some embodiments, as illustrated in FIG. 1C, a spacer 106 may be provided between the bent cylindrical body 102 and the hinge 130.

In some embodiments, the bent cylindrical body 102 and the plunger 104 may be operably connected by a hinge 130 positioned at an intersection of a radius of the cylindrical body 102 and the plunger 104. The hinge 130 may be a mechanical hinge including separate parts on the body 102 and the plunger 104 that operably connect to create the hinge. Such mechanical hinges include, but are not limited to, barrel hinges, butt and mortise hinges, pivot hinges, and the like. In particular embodiments, the mechanical hinge may be an open barrel syringe including a sleeve portion 132 having a substantially C-shape defined on either a portion of the cylindrical body 102 or the plunger 104. FIG. 1A shows the sleeve 132 defined on a portion of the cylindrical body. The other of the cylindrical body 102 or plunger 104 may include a solid shaft 134 sized to fit within the sleeve 132 and rotate within the sleeve 132 to provide a rotation about a fixed axis of rotation. As depicted in FIG. 1A, the solid shaft 134 is defined at an end of a plunger arm 126 that connects the hinge 130 to the proximal end of the rod 122.

In other embodiments as illustrated in FIG. 1D and FIG. 1E, the hinge 130a may be molded from the material used to create the body 102 and the plunger 104 such that the bent syringe may be a single body molded 12,13 to include both the body 102 and the plunger. The molded hinge 130a may be provided at the mid section of a body-plunger assembly 12,13 and can include a notch 135 that allows the body-plunger assembly 12,13 to be folded at the notch 135. When folded as illustrated in FIG. 1E, the plunger 104 may be operably received by the body 102 to allow the syringe to be operated as discussed above with regard to bent syringes having a mechanical hinge.

The bent cylindrical body 102 may include any number of additional elements associated with syringes including, for example, markings or graduations (not shown) on a surface of the bent cylindrical body 102 showing the volume of the syringe from the distal end 114 of the bent cylindrical body 102 to the graduation marking. In some embodiments, the bent cylindrical body 102 may include one or more fitting 118 located at an outlet port 116 of the bent cylindrical body 102, and the distal end 114 of the bent cylindrical body 102 may have a generally conical shape 117 with the outlet port 116 being centered within the conical portion. The fitting 118 may be any type of fitting known in the art including, but not limited to, luer fittings, screw-type fittings, pressure fittings, and the like. In some embodiments as illustrated in FIG. 1D and FIG. 1E, the bent cylindrical body 102 may include an integral needle 119 at the outlet port 116 that is molded with the cylindrical body 102. In other embodiments, an integral needle 119 may be fixedly attached to the cylindrical body 102 after the cylindrical body has been molded using, for example, an adhesive.

The bent syringed 10,11 of various embodiments can be stand-alone devices that are configured to be used by hand. In such embodiments, the bent cylindrical body 102 may include one of more elements that are positioned and configured to allow a user to grasp the bent cylindrical body during use. For example, the bent cylindrical body 102 may include one or more flanges, contoured portions, abrasive surfaces, and the like and combinations thereof. The plunger 104 may include a flat surface associated with the plunger arm 126 that is configured to be manipulated by a user. For example, portions of the plunger arm surrounding the piston 122 meets the plunger arm 126 may be contoured or include an abrasive surface, or extensions may be provided to increase the width and length of this section providing a thumb rest. In still other embodiments, the plunger 104 may include a handle extending from the rod or plunger arm to facilitate grasping by the user and movement of the plunger though the bent cylindrical body 102.

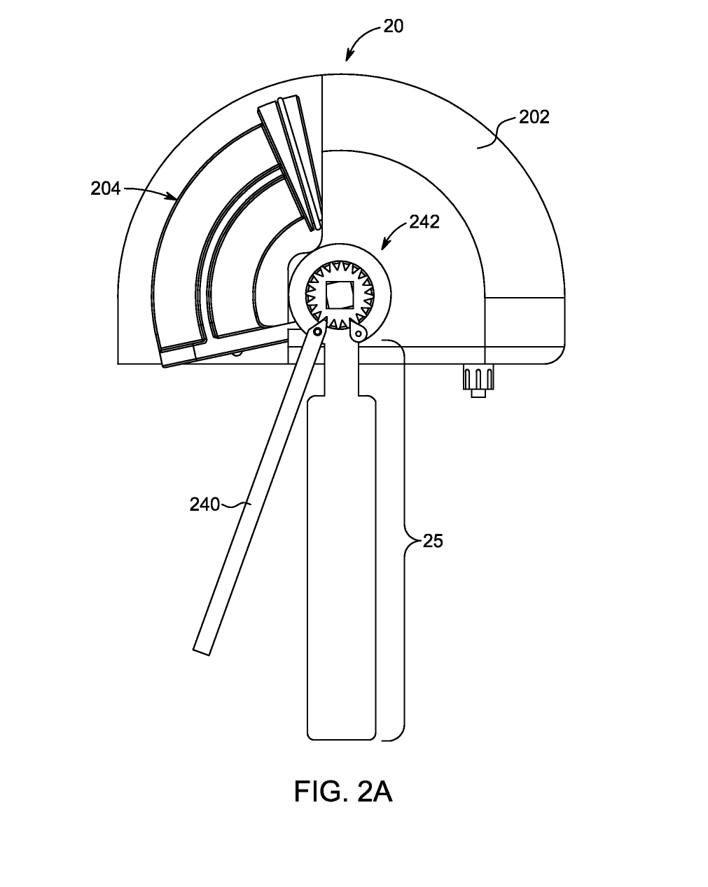

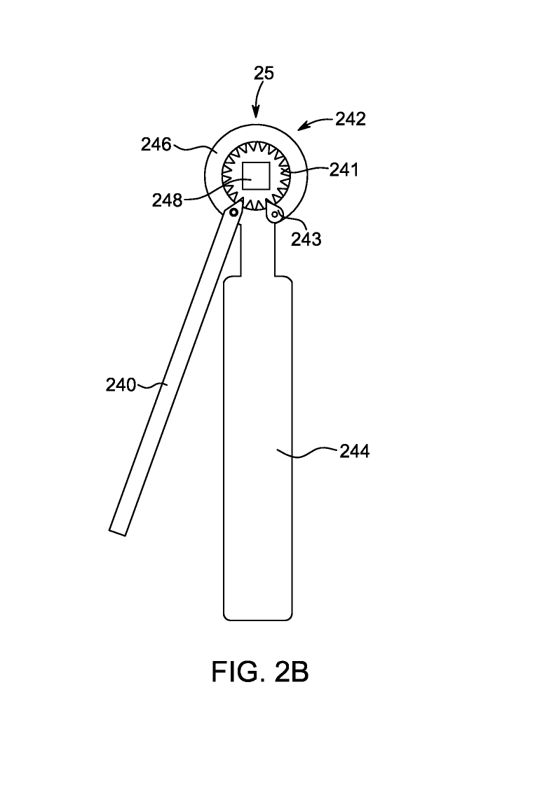

In particular embodiments, the bent syringe 10,11 may include a mechanical means for moving the plunger 104 through the bent cylindrical body 102. For example, in some embodiments, as illustrated in FIG. 2A the syringe 20 may include a handle 25 having ratchet 242 and a trigger 240 mechanism for advancing the plunger 204 through the bent cylindrical body 202 by hand. The handle 25 is illustrated more specifically in FIG. 2B, which shows a handle 25 including a grip 244 fixedly attached to a housing 246. A spindle 248 may be fixedly attached to a gear 241 and rotationally attached to the housing 246. The trigger 240 may be attached to the gear 241 such that movement of the trigger 240 toward the grip 244 causes the gear 241 to rotate within the housing. A pawl 243 may be attached to the housing 246 that stops the gear 241 from rotating backward after the trigger 240 has caused the gear 241 to rotate forward. The spindle 248 may be designed and configured to engage a portion of the hinge 242 to cause rotation of the hinge 242 and causing the plunger to advance through the bent cylindrical body. The ratchet system 242 may include additional elements such as springs additional gearing, and housing elements. For example, the ratchet system may include one or more springs positioned to allow the trigger to be returned to a starting position when pressure is released from the trigger.

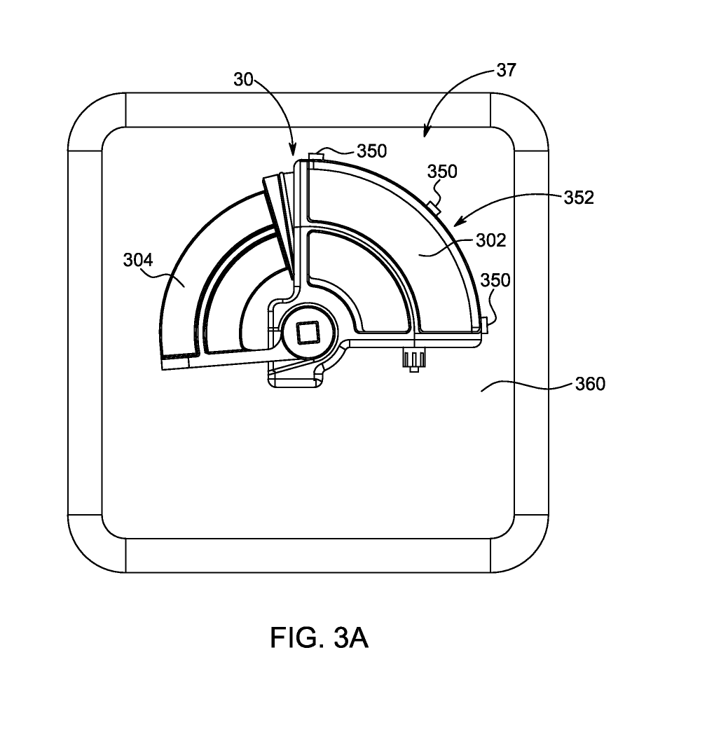

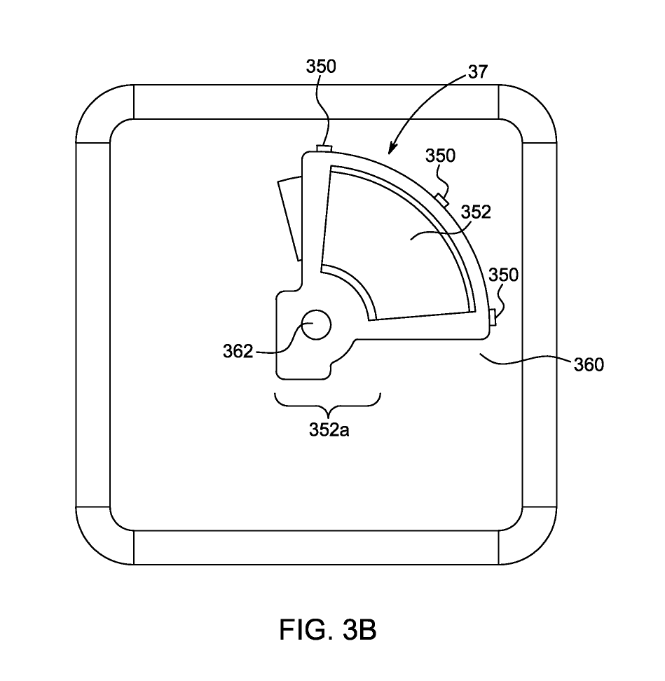

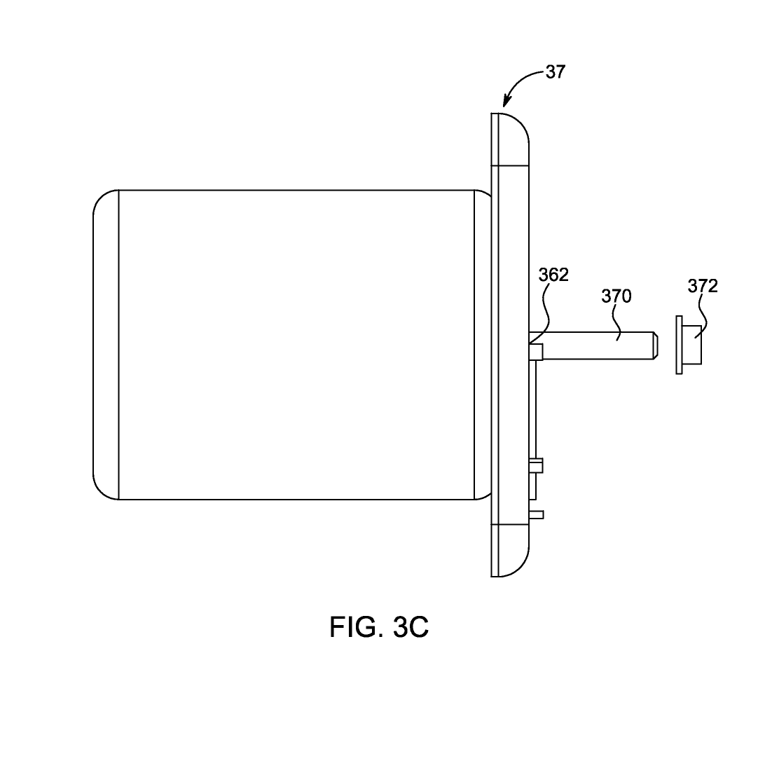

In some embodiments as illustrated in FIG. 3A and FIG. 3B, the bent syringe 30 may include a mounting system 37 that reversibly attaches to the bent syringe 30. The mounting system 37 may be attached to a larger device or the mounting system may include various elements for manipulating the bent syringe by hand. In various embodiments, the mounting system 37 may include clips 350, pins, clasps, screws, depressions 352, or other attachment means, or combinations thereof, positioned to securely attach the bent syringe 30 to the mounting system 37. For example, in some embodiments, the mounting system may include clips 350 positioned to engage the bent cylindrical body 302 while allowing the plunger 304 to advance and retreat freely within bent cylindrical body 302. The mounting system may be designed to attach to one or more sides of the bent syringe. For example, in some embodiments, the mounting system may attach to one side of the bent syringe and in other embodiments, the mounting system may attach to at least two sides of the bent syringe or completely encase the bent syringe. Thus, in certain embodiments, the bent syringe may be removably inserted into a mounting system that acts as a syringe holder.

In embodiments in which the mounting system is designed to allow the bent syringe to be manipulated by hand, the mounting system may include flanges, handles, contoured and/or textured surfaces, and the like, configured and arranged to allow a user to grasp and manipulate the bent syringe, and in some embodiments, the mounting system may be associated with a mechanical means for advancing the plunger such as the ratchet system described above.

In particular embodiments, the mounting system 37 may be fixedly attached to a device, and the bent syringe 30 may be removably received by the mounting system 37. In some embodiments, the mounting system 37 may include a plate 360 configured to attach to the housing of a device, and one or more clips, pins, clasps, screws, depressions, or other attachment means, or combinations thereof 350,352, attached to the plate 360 for attaching the bent syringe 30 to the mounting system 37 and for properly aligning the bent syringe 30 on the device. In other embodiments, the mounting system may be an integral part of the housing such that one or more clips, pins, clasps, screws, depressions, or other attachment means, or combinations thereof may be attached directly to the housing and positioned to attach the bent syringe to the housing.

In some embodiments as illustrated in FIG. 3B, the mounting system may include an opening or hole 362 through which a spindle can pass. In various embodiments, the opening or hole 362 may be positioned to correspond with the position of the hinge of the bent syringe 352a as illustrated by the depression 352, such that the spindle can pass through the opening 362 to engage the bent syringe at the hinge. Rotational movement of the spindle will allow the hinge to open and close thereby allowing materials to be introduced into or expelled from the syringe by movement of the plunger.

FIG. 3C shows a profile of the mounting system illustrating the spindle 370 passing through the opening or hole 362. The spindle 370 may be a spindle of any design. For example, in some embodiments, the spindle 370 may be shaped to engage a correspondingly shaped hinge on the bent syringe. Various exemplary cross sectional spindle shapes include, but are not limited to, triangular, square, pentagonal, octagonal, and the like and various star shaped cross sectional shapes. The hinge of the bent syringes designed to engage these shaped spindles will have a corresponding triangular, square, pentagonal, octagonal, and the like or a star shape. In particular embodiments, the spindle may be cylindrical. In some embodiments, cylindrical spindles may be received by a pressure fitting that allows stable attachment of the syringe to the spindle and transfer to the rotation of the spindle to the bent syringe. In other embodiments, a portions of the spindle may protrude beyond the bent syringe, and a further attachment device such as, for example, a screw cap 372, cotter pin, or the like may attach to the protruding portion of the spindle to attach the spindle 370 to the syringe and allow transfer of rotation of the spindle 370 to the syringe.

In certain embodiments, the spindle may be received by a one-way clutch mechanism that allows rotation of the spindle to produce forward movement of the plunger advancing the plunger into the syringe body, but does not allow reverse rotation of the spindle to withdrawal the plunger from the syringe body. In embodiments in which a clutch is employed, the plunger may be advanced by rotation of the spindle, but will remain in the advanced, forward position when rotation of the spindle is reversed. This may reduce the likelihood of accidental reversal causing the fluids expelled from syringe to be sucked back into the syringe body as the plunger withdrawals. In some embodiments, the clutch mechanism may include a stop that allows both forward and reverse rotation of the spindle to be transmitted to the plunger allowing one-way and two-way movement to be controlled by the user.

In particular embodiments, the bent syringe may be mounted on the spindle alone. In such embodiments, the spindle may engage the bent syringe with sufficient hold to maintain the position of the bent syringe on the device without the use of clips or other attachment means. Thus, in some embodiments, a device may include spindle protruding from a housing onto which a bent syringe may be mounted. As discussed above, such spindles may be of any design, and the spindle may hold the syringe by pressure, shape, or using a separate attachment means such as a screw cap, cotter pin, or the like.

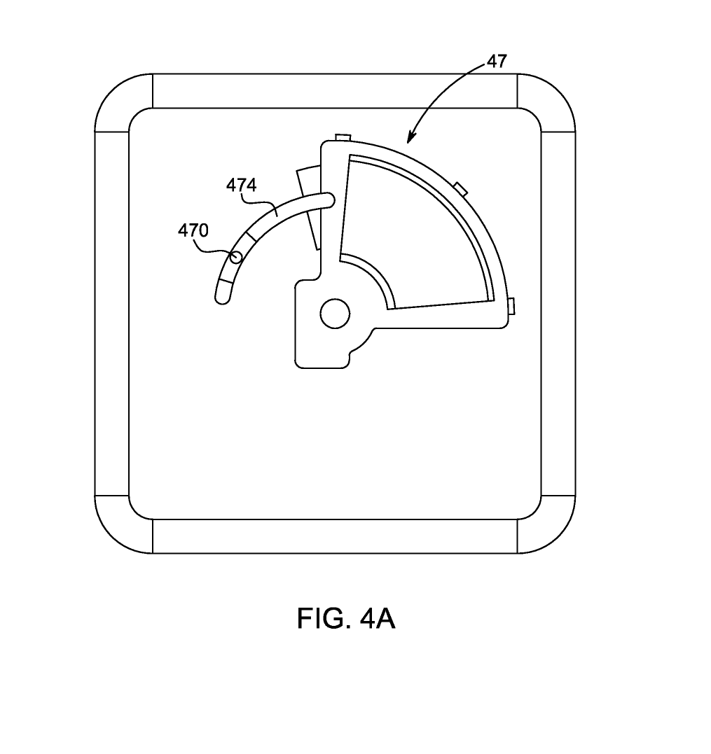

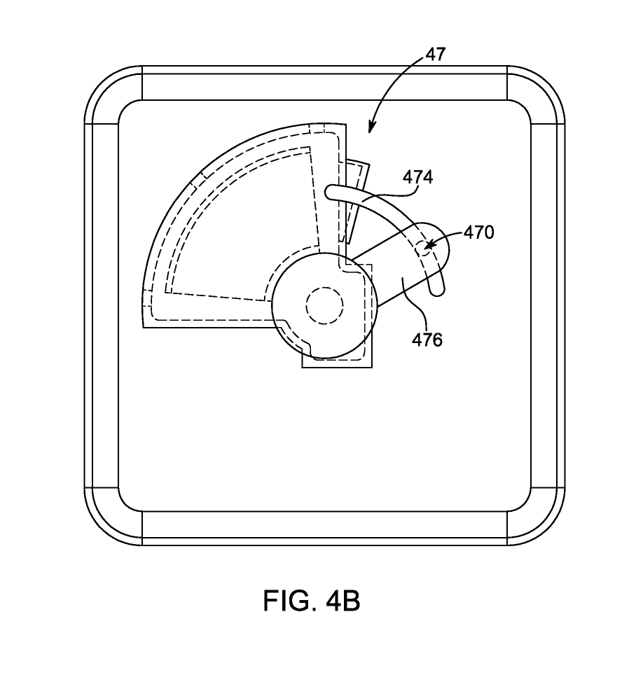

In certain embodiments as illustrated in FIG. 4A, the mounting system 47 may include one or more grooves 374 aligned with the plunger portion of the bent syringe. In such embodiments, a spindle 370 may pass through the one or more groove 374 and be received by a portion of the plunger arm. As illustrated in FIG. 4B, the spindle 370 may be fixedly attached to a rotatable gear or arm 376 on a side of the mounting system 37 opposite the bent syringe that is configured to move the spindle 374 through the groove 376 and urge the plunger through the syringe body. In some embodiments, the spindle 374 may removably attach to the plunger arm by connecting to the syringe arm using, for example, a mounting cavity or orifice incorporated into the plunger arm to allow the spindle and associated gearing to control the advancement and withdrawal of the plunger in the syringe body. In other embodiments, the spindle may contact the plunger arm without connecting to the plunger arm thereby allowing movement of the spindle to advance the plunger through the syringe body by forward movement of the spindle against the plunger arm but allowing the plunger to remain in place when movement of the spindle is reversed.

In some embodiments, the mounting system may be capable of rotating allowing the bent syringe to assume a number of positions. For example, in some embodiments, the mounting system may allow the bent syringe to rotate from a position in which the outlet and fitting associated with the bent cylindrical body face downward to a position in which the outlet and fitting face upward, and in other embodiments, the bent syringe may be rotated such that the outlet and fitting are move from a lateral facing position to an upward facing position. In embodiments including a plate mounting system, the plate may include a rotating mount onto which the bent syringe is attached. Thus, a portion of the plate may be securely mounted on the device housing, while the bent syringe is capable of rotating on the rotating mount. In other embodiments, the housing may include a rotating mount and the plate may be attached to the rotating plate. In either such embodiments, the rotating mount may be freely rotatable, or the rotating mount may include two or more catches or stops positioned to halt rotation of the bent syringe at particular positions, such as an upward facing position and a downward or lateral facing position. In still other embodiments, the rotating mount may include a ratchet system that allows rotation to be halted at a number of positions. In some embodiments, the rotating mount may be associated with a motor or other mechanical means for rotating the rotating mount, and in other embodiments, the rotating mount may be manually operated.

In embodiments in which the bent syringe mounts directly onto the spindle, rotation of the bent syringe may be effectuated by disengaging a motor or other mechanical means for applying rotation movement to the plunger, or the motor or mechanical means for applying rotational movement to the plunger may allow for free rotation of the bent syringe when not engaged. As above, rotation of the bent syringe about the spindle may be free rotation allowing the syringe to rotate to any position, or two or more stops may be associated with the bent syringe to halt rotation at particular positions. Such stops may be associated with the syringe, for example, one or more extension may project from the bent cylindrical body that are positioned to engage a groove in the housing that causes the syringe to stop rotation when an end of the groove is reached. The groove may further include teeth or other projections that can engage the extensions and stop rotation at various positions along the groove. In other embodiments, projections from the housing that are positioned to engage the syringe and stop rotation. For example, a first projection from the housing may engage a distal portion of the bent cylindrical body and stop rotation when the outlet and fitting are in a downward position and a second projection may engage a proximal portion of the bent cylindrical body when the outlet and fitting are in a upward facing position.

In operation, rotation of the bent syringe may allow the syringe to be loaded while mounted on the device. For example, the bent syringe may be rotated such that the outlet and fitting are facing upward during or after the syringe has been loaded allowing air bubbles to surface near the outlet and fitting. By depressing the plunger while the outlet and fitting are facing upward, such air bubbles may be expelled from the syringe. The syringe may then be rotated to a position that facilitates delivery of the substance loaded into the syringe body, for example, the outlet and fitting facing downward or laterally.

Embodiments of the invention further include devices configured and designed to incorporate the bent syringes and mounting systems described above. In general, such devices may include at least one drive motor capable of producing rotational movement and a spindle operatively connected to the drive motor and capable of rotating by action of the drive motor. The spindle may be configured to reversibly connect to the hinge of a bent syringe and transfer rotational movement of the drive motor to the hinge and cause the plunger arm to advance or withdrawal from the syringe body by action of the drive motor. In some embodiments, the spindle may extend directly from the drive motor, and in other embodiments, the spindle may be operably connected to the drive motor through gears, belts, cables, chains, and the like or other means for transmitting rotation force from one device to another. In particular embodiments, a circular or semicircular gear or an arm may be fixedly attached to a first spindle extending from the drive motor, and a second spindle designed to operably connect to the plunger arm of a bent syringe may be positioned along a radius of the gear or at along the arm. This arrangement may allow the drive motor to provide arced movement of second spindle, which can be used to advance or withdrawal a plunger in a bent cylindrical body by rotational movement of the drive motor.

In some embodiments, the bent syringe may attach to the device using a mounting system designed to contact and hold at least the bent cylindrical body portion of the bent syringe. In general, such a mounting system may be disposed between the drive motor and any gears, belts, cables, chains, and the like associated with the drive motor and the bent syringe when the bent syringe is mounted on the device. In some embodiments, the mounting system may be attached to a housing of the device, and in other embodiments, the mounting system may attach to the drive motor or a portion of the drive motor. As discussed above, the mounting system of various embodiments may include any number of holes, orifices, or grooves designed and configured to allow passage of the spindle through the mounting system and facilitated proper alignment of the bent syringe for attachment to the spindle. In some embodiments, the holes, orifices, or grooves may be designed and configured to associate with corresponding holes, orifices, or grooves in a housing that are also positioned to allow passage of the spindle. The mounting system may further include any number of clips, pins, clasps, screws, depressions, or other attachment means, or combinations thereof, positioned to securely attach the bent syringe to the mounting system and the device. The size of the mounting system may vary among embodiments and may dictate the number and type of attachment means associated with the mounting system. For example, a small mounting system having 1, 2, 3, or 4 clips, pins, clasps, and the like may be utilized one devices or portions of devices having a housing with a small surface area. In embodiments in which the mounting system is attached to a device housing having a large surface area, a greater number of attachment means may be utilized. Of course, even mounting systems on large surface area housings may include a small number of attachment means.

The devices of various embodiments may include a drive motor or other mechanical means for rotating the bent syringe. The type of motor is not limited and can include any type of motor known and used in the art. In general, the motor may be capable of rotating a spindle with sufficient force to cause the plunger to rotate within the bent cylindrical body of the bent syringe and force the contents of the syringe through the distal orifice. In other embodiments, the motor may provide sufficient force to push the expelled contents of the syringe through a fluid pat set as described below. The motors of embodiments may be controllable by the user such that the user can control the rotation of the spindle during operation or using preprogrammed controls provided in a computing device or processor associated with the device. For example, in some embodiments, a button may activate the motor causing the spindle and plunger to rotate until the button is deactivated. In other embodiments, a button may be depressed that causes the motor to activate for a predetermined amount of time causing a specific amount of rotation to occur. In still other embodiments, a computing device or processor may activate the motor for specific time periods rotating the spindle and plunger causing movement of the plunger various distances at particular predetermined times as a programmed procedure is carried out.

The devices of embodiments may include any number of additional features including, for example, a user interface, buttons, dials, gauges, containers for fluids and/or waste materials, user control mechanisms for controlling the drive motor and/or bent syringe, handles, wheels or casters, or any other feature used in medical devices.

In some embodiments, the bent syringe may include a pressure monitoring device attached to the outlet port or fitting associated with the outlet port. In some embodiments, the pressuring monitoring device may be attached to a user interface to notify the user of the pressure within the bent cylindrical body and the pressure with which fluid is expelled from the bent cylindrical body. In certain embodiments, the pressure monitoring device may be operably connected to the drive motor through a relay that causes the drive motor to slow or stop when, for example, the pressure inside the bent cylindrical body reaches a maximum or increases pressure within the bent cylindrical body when a minimum pressure is reached.

In certain embodiments, the devices may further include a fluid path set providing tubing into which the bent syringe can expel the contents of the syringe body. Such fluid path sets may include one or more lengths of tubing that are capable of connecting to a fitting on the bent syringe at a first end and a discharge means at a second end. The discharge means may be a fitting, a another device, or a container, and in particular embodiments, the discharge means may be a hypodermic needle configured and designed to allow administration of the contents of the bent cylindrical body to a patient. The fluid path set may further include junctions and valves useful for, for example, partitioning the expelled contents of the bent syringe, combining additional components with the contents of the bent syringe, modifying the delivery path of the expelled contents, transporting the contents to a waste receptacle, or the like.

In some embodiments, a device may include two or more bent syringes. The device may be configured to allow simultaneous, sequential, or independent delivery of fluids from the two or more bent syringes. The two or more bent syringes can be associated with individual drive motors, or in other embodiments, the two or more syringes may be associated with the same drive motor in either a side by side configuration in which both bent syringes are on the same spindle on the same side of the drive motor or the two or more syringes may be on the same or different spindles on opposite sides of the drive motor. The device may be designed to deliver the same material from the two or more bent syringes. In other embodiments, the device may be designed to deliver different materials from the two or more bent syringes, and the fluid to be delivered can be delivered through the same fluid path to facilitate delivery to the patient or different fluid paths. In some embodiments, the fluids may be combined or mixed in the fluid path before delivery, and in other embodiments, the fluids may be delivered in separately and/or sequentially.

1. A syringe, comprising:
a bent cylindrical body bent about a radius, the bent cylindrical body having a proximal open end and a distal conical end, the conical end comprising at least one discharge outlet; and
a plunger having a curvature substantially similar to the curvature of the bent cylindrical body configured and arranged to be received by the proximal open end of the bent cylindrical body and movably disposed within at least a portion of the bent cylindrical body.

2. The syringe of claim 1, wherein the radius is from about 30° to about 180°.

3. The syringe of claim 1, wherein the radius is from about 45° to about 100°.

4. The syringe of claim 1, wherein the radius is about 90°.

5. The syringe of claim 1, further comprising a hinge disposed at an intersection of a radius of the bent cylindrical body and a radius of the plunger and operably connecting to the bent cylindrical body and the plunger.

6. The syringe of claim 5, wherein the plunger further comprises a plunger arm connecting the hinge and a proximal end of the plunger.

7. The syringe of claim 5, further comprising at least one drive motor operably connected to one of the bent cylindrical body or the plunger.

8. The syringe of claim 5, further comprising at least one drive motor operably connected to each of the bent cylindrical body and plunger.

9. The syringe of claim 5, further comprising a spindle received by the hinge and operably connected to one of the bent cylindrical body or the plunger.

10. The syringe of claim 9, further comprising a drive motor operably connected to the spindle.

11. The syringe of claim 1, wherein the bent cylindrical body is rigid.

12. The syringe of claim 1, wherein the bent cylindrical body is composed of a material selected from the group consisting of glass, metal, plastic, and combinations thereof.

13. The syringe of claim 1, wherein the bent cylindrical body further comprises one or more flanges formed on the bent cylindrical body.

14. The syringe of claim 1, further comprising a mounting system capable of reversibly attaching to the bent syringe.

15. The syringe of claim 1, further comprising one or more flanges configured and arranged to allow the bent cylindrical body to be grasped by a user.

16. The syringe of claim 1, wherein the discharge outlet comprises a fitting.

17. The syringe of claim 16, wherein the fitting is selected from the group consisting of pressure fittings, luer fittings, and screw fittings.

18. The syringe of claim 1, wherein the bent cylindrical body further comprises one or more markings, wherein each marking corresponds to a volume of liquid contained within the bent cylindrical body when the plunger is positioned at the marking.

19. The syringe of claim 1, wherein the plunger further comprises one or more flanges.

20. The syringe of claim 1, wherein the plunger further comprises a stopper at a distal most end of the plunger, the stopper having a circumference substantially identical to a circumference of an inner lumen of the bent cylindrical body.

21. The syringe of claim 1, wherein the plunger further comprises a flange at a proximal end of the plunger.

22. The syringe of claim 5, further comprising a mounting system comprising a plate removeably attached to the bent cylindrical body by one or more clips, pins, clasps, screws, depressions, and combinations thereof.

23. The syringe of claim 22, wherein the plate further comprises one or more holes configured to receive a spindle therethrough.

24. The syringe of claim 1, further comprising a hinge and a spindle, wherein the hinge comprises a first portion of the hinge operably connected to the bent cylindrical body and a second portion of the hinge operably connected to the plunger, the spindle being operably connected to one of the first portion of the hinge or the second portion of the hinge.

25. The syringe of claim 24, further comprising a drive motor operably connected to the spindle and being capable of providing rotational force to the spindle.

26. A device comprising:
a syringe, comprising:
a bent cylindrical body bent about a radius and having a proximal open end and a distal conical end, the conical end comprising at least one discharge outlet;
a plunger having curvature substantially similar to the curvature of the bent cylindrical body and being configured and arranged to be received by the proximal open end of the bent cylindrical body and movably disposed within at least a portion of the bent cylindrical body; and
a hinge operably connected to the bent cylindrical body and the plunger, the hinge being disposed at an intersection of the radius of the bent cylindrical body and a radius of the plunger;
a spindle configured and designed to be received by the hinge and operably connected to one of the bent cylindrical body or the plunger; and
a drive motor operably connected to the spindle and being capable of providing rotational force to the spindle.

27. The device of claim 26, wherein the syringe is removable from the device.

28. The device of claim 26, wherein the discharge outlet comprises a fitting selected from the group consisting of pressure fittings, luer fittings, and screw fittings.
